(19) United States
(12) Patent Application Publication
(10) Pub. No.: US 2016/0113990 A1
(43) Pub. Date: Apr. 28, 2016

(54) HERBAL FORMULATION OF AN EDIBLE ORAL TURMERIC, ITS DERIVATIVES & OTHER INGREDIENTS FOR THE MANAGEMENT OF INFLAMMATION AND CHRONIC PAIN

(71) Applicant: Shelia Cockburn, New York, NY (US)

(72) Inventor: Shelia Cockburn, New York, NY (US)

(21) Appl. No.: 14/923,400

(22) Filed: Oct. 26, 2015

Related U.S. Application Data
(60) Provisional application No. 62/069,795, filed on Oct. 28, 2014.

(51) Int. Cl. A61K 36/9066; A61K 36/324; A61K 31/385; A61K 31/12

(52) U.S. Cl. CPC A61K 36/9066; A61K 31/12; A61K 36/324; A61K 31/385

(57) ABSTRACT

The Invention provides a herbal formulation for managing inflammation in Mammals comprising of a) an active ingredient which is a combination of Turmeric, Curcumin, Boswellia Serrata, b) fatty acid or lipid component to increase the stability, absorption and bioavailability of Turmeric and Boswellia and c) common edible taste masking ingredient in a quantity sufficient to mask the taste of other ingredients. In addition to reducing inflammation, the herbal formulation also provides antioxidant benefits and promotes healthy joint function.

### BACKGROUND OF THE INVENTION

The invention relates to the field of herbal formulations for management of health related conditions. More specifically the invention relates to an herbal/organic oral edible composition having anti-inflammatory, anti-oxidant and anti-proliferative properties. The invention further relates to methods for utilizing such compositions for the treatment and management of inflammation and pain in mammals like Humans, Horse, Cat, Pigs, Dog, etc.

Proven studies are well documented on the benefits of omega-3 fatty acids (EPA, DHA, ALA and DPA) on normalizing metabolism, the reduction of inflammation, and the prevention of a number of diseases, and, such as cardiovascular, mental health, wellness and ageing. The availability and use of high quality omega-3 oils, alone or in other products, has increased dramatically and is often recommended by doctors to their patients. Similarly, substances high in antioxidants, which react with free radicals, are well know to prevent damage to mammalian cells and thereby slow the ageing process. Finally, anti-proliferative substances are known to retard malignant cell growth into surrounding tissue; which may prevent cancer.

Free radicals are atoms that, for whatever the cause, start a chain reaction within the body that can ultimately damage DNA or cell membranes leading to illness and disease. Inflammation, within the body, is a by-product of an abnormally high quantity of free radicals. If inflammation remains untreated, known as a precursor to many illnesses and diseases, is believed to facilitate the ageing process and to blossom into conditions such as arterial hypertension, osteoarthritis, arthritis, Alzheimer's, and several types of cancer.

Turmeric, as well as its derivatives and analogs, is a spice wherein over 80% is produced in Asia, specifically India, and is known as a traditional medicine used to treat ailments byway of topically, orally and by inhalation. Curcuminoids is a natural product from the turmeric, which comes from the root of Curcuma longa.

Curcuminoids encompasses 2% to 5% of turmeric root and comprises one or more of a mixture of curcumin, demethoxycurcumin, turmerones, extracts of the essential oil of curcuma longa, bisdemethoxycurcumin, and other derivatives and/or analogs, which are thought to be the main active components in treating ailments. Many studies, on non-human mammals such as mice, have used curcumin or curcuminoids to test its anti-proliferation properties against tumors such as in the colon, skin, stomach and breasts. Other trials have shown that curcuminoids can reduce oxidized proteins and inflammatory cytokines interleukin-1.beta; which may offer benefits in protecting proper brain functioning as curcumin has clearly demonstrated that, since it is highly lipophilic, it can easily cross the blood-brain barrier in sufficient quantities to aid in healing. Further other studies in mammals have shown there are anti-diabetic and anti-microbial properties. In addition, curcuminoids has been shown to display anti-inflammatory, potent anti-oxidant and free radical scavenging abilities. Prior art has shown that curcumin shows promise in being able to reverse certain types of disease progression in areas that also concern the reversal, the elimination or the reduction of inflammation and chronic pain and curcumin shows improved performance when combined with an enhancer such as black pepper or piperine of up to 20 mg per dosage or per day.

Most studies have used curcuminoids in pill, gelcap, capsule, tablet or through injections in efforts to decrease bioavailability issues or increase bioavailability. With all its beneficial effects, presently there are bioavailability problems associated with the oral delivery of curcuminoids. It has been found that less than 1% of oral curcuminoids are absorbed into the bloodstream because curcuminoids are not readily absorbed in the human digestive tract and suffers intestine-based metabolism. Further, the liver and kidney metabolize even the small amount that enters the bloodstream. Therefore, only very small amounts of curcuminoids, most studies orally administered in pill, capsule or injectable forms, are available in plasma to reach brain tissue. Prior bioavailability studies reveal that ingestion of about 6-8 grams of curcuminoids produced a peak serum level in the range of about 0.51-1.77 μM. In the brain, it appears that concentrations of up to 0.4 μM have been detected when injectable curcuminoids were used. Prior studies have shown that a steady peak range of concentrations of between 0.1 to 1 μM may be needed to see at least some on-going therapeutic benefits in the brain, but other studies have shown that therapeutic benefits were detected in a mammalian body even when curcuminoid concentrations became hard to measure over time. Furthermore, it has been reported that oral doses of curcuminoids of about 6-8 grams per day may cause headaches and diarrhea in some recipients.

Another traditional herb, also called boswellia or frankincense, where studies have shown the resin is beneficial as an analgesic; in the elimination of inflammation, and more specifically the elimination of a pro-inflammatory enzyme 5-lipoxygenase; and as a treatment of other chronic ailments such as arthritis, the control of coronary plaque and the control of blood lipids.

Boswellia is generally taken orally as a capsule, tablet or liquid extract. The recommended dosage is based on traditional history or as know in the art of 150-400 mg up to three times a day.

Another important aspect to note is that nutrients carried in plasma, and ultimately to the brain to support its functioning, have been shown to be best transported in conjunction with high amount of lipids and fatty acids. The blood brain barrier supports nutrients through lipid solubility or specific transporter proteins.

Another important aspect to note down is that the unpleasant taste or odor of herbal formulations is reason for which many people avoid them. The unpleasant taste or odor is contributed by ingredients other that organic acids. The taste masking of such formulation is an important component when it comes to making of consumer acceptable herbal formulations.

Another important aspect shown by prior art is that self-administered compositions in pill, gelcap or injectable form has low adherence rates. It is well documented that up to 50% of medications are not taken as prescribed and 20% or more persons fail to even get the prescription filled. This is often due to: the high cost of medicine; the stigma of appearing abnormal or mentally/physically unstable; the associated medication side effects & forgetfulness. The administration method of a formulation is an important component to ensure the delivery of a health benefit, maintain use and to maintain compliance.

Hence there is a need to develop an herbal formulation, which will overcome the above problems mentioned in the prior art related to medical compositions and herbal formulations.

### SUMMARY OF THE INVENTION

The formulation of the present invention comprising of a therapeutically effective concentration of Turmeric, Curcumin, Boswellia Serrata, at least on fatty acid or lipid component in a concentration sufficient to increase the stability, absorption and bioavailability of Turmeric and Boswellia combined with common edible taste masking ingredient in a quantity sufficient to mask the taste of other ingredients. The herbal or organic formulation comprises of a therapeutically effective amount of Turmeric, Curcumin, Boswellia Serrata or combination thereof for controlling inflammation and to reduce chronic pain. The fatty acid or lipid component of the formulation used to achieve enhanced absorption, increasing the bioavailability, increase retention can be selected from the group of alpha Lipoic acid, R-Lipoic Acid (RLA), R-Dihydrolipoic Acid (R-DHLA), etc or combination thereof. The taste masking component of the formulation can be selected from the group of Soy lecithin, Honey, Chocolate, Nut butters (almond/cashew/macadamia), Fish oil, Grape Juice, Pepper or other commonly available ingredients or combination thereof. Other known materials which can be used are polysaccharides, hydrocolloids and collagens and glycoproteins and such combinations mixed with natural available solvents, such as water, liquid concentrates and other fluids used with dissolving methods known in the art with binding agents such as a starch, fiber, lecithin etc.; which can be effectively used as oral therapeutic agents at doses up to and including the tolerable limits for each, or in combination, to ingredient consumption limits. The formulation can be taken daily, two through seven times per day in whole or in part, weekly or monthly and a combination thereof.

Solvent is used to dissolve all ingredients into the carrier that are solvent compatible whilst the curing carrier is used to hold all ingredients that are not directly compatible to solvent. Curing is then completed as required to set carrier.

The formulation of the present invention is convenient for self-administration and is safe for managing chronic disease and inflammation, which increases health and wellness.

The formulation can further consist of natural flavor, such as cocoa powder, chocolate, nut essences, etc or combination thereof.

### BRIEF DESCRIPTION OF THE DRAWING

No drawings;

### DETAILED DESCRIPTION OF THE INVENTION

As stated above, the present invention provides an orally administered herbal composition and a method of using the composition to reduce inflammation in animals, preferably humans, suffering from inflammation, pain or chronic pain.

The composition of this invention is composed of: therapeutic herbal ingredients like Turmeric, Curcumin, Boswellia Serrata, at least on fatty acid or lipid component in a quantity sufficient to increase the stability, absorption and bioavailability of herbal ingredients like Turmeric, Curcumin and Boswellia and at least one commonly used edible taste masking ingredient in a quantity sufficient to mask the taste of herbal ingredients.

The composition of this invention will contain “therapeutically effective amounts” of the therapeutic herbal ingredients recited above. As used herein with respect to each of the herbal extracts used in the composition of this invention, the term “therapeutically effective amount” refers to that amount of the extract, which will contribute to the inflammation-reducing ability of the composition.

The composition of this invention will contain “sufficient quantity” of fatty acid or lipid component. As used herein with respect to one or more fatty acid or lipid component used in the composition of this invention, the term “sufficient quantity” or “quantity sufficient” refers to that amount of the fatty acid or lipid component, which will improve the stability, absorption and bioavailability of the composition.

The composition of this invention will contain “sufficient quantity” of fatty acid or lipid component. As used herein with respect to one or more fatty acid or lipid component used in the composition of this invention, the term “sufficient quantity” or “quantity sufficient” refers to that amount of the fatty acid or lipid component which will improve the stability, absorption and bioavailability of the composition.

Preferably, the composition of this invention contains: a) About 250 mg to 1500 mg of Curcumin, 100 mg to 900 mg of Turmeric, 75 mg to 1000 mg of Boswellia Serrate. b) About 75 mg to 600 mg of alpha lipoic acid or any other fatty acid or lipid ingredient selected from the group of R-Lipoic Acid (RLA) and R-Dihydrolipoic Acid (R-DHLA) or combination thereof, c) At least one taste masking ingredient selected from the group of the nut butters (almond/cashew/macadamia), fish oil, pepper, pepper extract, chocolate, flavoring agent, ascorbic acid, lipoic acid or combination thereof.

In preferred embodiments, the composition of this invention further contains a pharmaceutically acceptable carrier or a pharmaceutically acceptable carrier that can also act as a preservative agent. As used herein, the term “pharmaceutically acceptable carrier” is meant to include one or more pharmaceutically suitable, inactive excipients, carriers, diluents, adjuvants, and lubricants. Non-limiting examples of inactive excipients, carriers, diluents, lubricants, and adjuvants which can be used in the composition of the present invention include: cellulose, substituted cellulose, calcium carbonate, dicalcium phosphate, starches, lactose, modified lbod starches, dextrose, calcium sulfate, magnesium carbonate, magnesium stearate, stearic acid, glycerin, vegetable oils, polysorbates, lecithin, silicium dioxide, food glaze, talc, croscarmeilose sodium, povidone, water and gelatin. Additional inactive excipients, carriers, diluents, lubricants and adjuvants which may be used with the active-ingredient composition of this invention are disclosed in the Handbook of Food Additives (CRC Press), which is incorporated by reference herein in relevant part.

The pharmaceutically acceptable carrier can be present in any conventional amount used in an orally administered compositions to ensure ease of use and compliance.

The present invention also provides a method for reducing inflammation in animals, more specifically mammals like Humans, Horse, Cat, Pigs, Dog, etc, preferably humans, suffering from inflammation or pain. The method of this invention involves the steps of: (1) providing the composition of this invention; and (2) orally or topically administering the composition to the animal in an amount and for a time period effective to reduce inflammation in the animal.

The taste masking agent of the formulation can be selected from the group of alcohol flavors like rum, nut butters like almond, cashew, peanut, macadamia, chocolate, Fish oil and/or fatty acids like cod or krill lecithin or soy lecithin, fiber (soluble and/or insoluble) like psyllium or inulin, meat flavor or broth like beef, pork, chicken, duck, lamb, sweetener like honey, sucrose or fructose nut oil like coconut, flax, walnut or sesame oil, juice like grape or apple juice, fruit flavors like strawberry, cherry, blueberry, calcium like calcium carbonate or calcium citrate or calcium phosphate, other flavors like egg and cheese or combination thereof.

The fatty acid or lipid component of the formulation can be selected from the group of nut butters like almond, cashew, peanut, macadamia, chocolate, Fish oil and/or fatty acids like cod, salmon or krill lecithin or soy lecithin, fiber (soluble and/or insoluble) like syllium or inulin, alpha lipoic acid, meat flavor or broth like beef, pork, chicken, duck, lamb, Vitamin D, nut oil like coconut, flax, walnut or sesame oil, fish powder like omega3 powder, plant powder like omega3 plant powder, calcium like calcium carbonate or calcium citrate or calcium phosphate, or combination thereof.

The binder component of the formulation, incorporates the solvent and the curing carrier, that can be selected from the group of pectin, gum Arabic, gelatin, known gelling agents, juice like grape or apple, carrageenan, fiber (soluble and/or insoluble) like psyllium or inulin, gum Arabic, lecithin or soy lecithin.

Various embodiments of the present invention are described by means of following non-limiting examples.

### EXAMPLE 1

### Variant of Edible Soft Chew

The composition of the Edible soft chew comprises of 780 mg of Turmeric, 900 mg Curcumin (95% std), 600 mg Boswellia (std acids), 300 mg of Alpha Lipoic Acid, 1 mg of pepper extract, 1100 mg of Fish Oil with taste masking agent, fatty acid, binder and pharmaceutically acceptable carrier.

### EXAMPLE 2

### Variant of Edible Soft Chew

The composition of the Edible soft chew comprises of 200 mg of Turmeric, 350 mg Curcumin (95% std), 200 mg Boswellia (std acids), 100 mg of Alpha Lipoic Acid, 0.5 mg of pepper extract, 600 mg of Fish Oil with taste masking agent, fatty acid, binder and pharmaceutically acceptable carrier.

1. A pharmaceutical formulation for management of inflammation and pain comprising of,
a) a mixture of therapeutically effective concentration of naturally occurring anti-inflammatory components like Turmeric, Curcumin and Boswellia Serrata;
b) at least one fatty acid or lipid ingredient in a sufficient amount to increase the bioavailability of Turmeric, Curcumin and Boswellia Serrata;
c) at least one taste masking ingredient in a sufficient amount to mask the taste of a naturally occurring anti-inflammatory substances of (a).

2. A pharmaceutical formulation according to claim 1 wherein the concentration of Turmeric is 100 mg-900 mg.

3. A pharmaceutical formulation according to claim 1 wherein the concentration of Curcumin is 250 mg-1500 mg.

4. A pharmaceutical formulation according to claim 1 wherein the concentration of Boswellia Serrate is 75 mg-1000 mg.

5. A pharmaceutical formulation according to claim 1 wherein the fatty acid or lipid ingredient is selected from the group of Alpha lipoic acid, R-Lipoic Acid (RLA) and R-Dihydrolipoic Acid (R-DHLA) or combination thereof.

6. A pharmaceutical formulation according to claim 1 or 5 wherein the fatty acid or lipid ingredient is alpha lipoic acid.

7. A pharmaceutical formulation as in any one of the preceding claims wherein the concentration of alpha lipoic acid is in the range of 75 to 600 mg

8. A pharmaceutical formulation according to claim 1 wherein the taste masking agent is selected from the group of the nut butters (almond/cashew/macadamia), fish oil, pepper or pepper extract, fatty acid, chocolate, flavoring agent, ascorbic acid, sweetenters, meat flavors or combination thereof.

9. A pharmaceutical formulation according to claim 1 or 8 wherein the taste masking agent is chocolate.

10. A pharmaceutical formulation according to claim 1 or 8 wherein the taste masking agent is a fruit flavor.

11. A pharmaceutical formulation according to claim 1 or 8 wherein the taste masking agent is a meat flavor.

12. A pharmaceutical formulation according to claim 1 wherein the said formulation may optionally contain Binder.

13. A pharmaceutical formulation according to claim 1 wherein the said formulation may optionally contain a preservative agent.

14. A pharmaceutical formulation as in any one of the preceding claims wherein the taste masking agent is a flavoring agent.

15. A pharmaceutical formulation as in any one of the preceding claims wherein the said formulation may optionally contain a binder.

16. A pharmaceutical formulation according to claim 5 or 8 wherein the said formulation may optionally contain a preservative agent.
